(19) United States
(12) Patent Application Publication
(10) Pub. No.: US 2012/0276520 A1
(43) Pub. Date: Nov. 1, 2012

(54) ASSAY FOR MUTATIONS IN STEM CELLS AND THEIR DERIVATIVES

(75) Inventors: John R. McCarrey, San Antonio, TX (US); Mary Pat Moyer, San Antonio, TX (US)

(73) Assignee: THE BOARD OF REGENTS OF THE UNIVERSITY OF TEXAS SYSTEM, Austin, TX (US)

(21) Appl. No.: 13/320,710

(22) PCT Filed: May 14, 2010
(86) PCT No.: PCT/US2010/034927

§ 371 Date: Jul. 6, 2012

Related U.S. Application Data
(60) Provisional application No. 61/178,705, filed on May 15, 2009.

(51) Int. Cl. C12Q 1/70

(52) U.S. Cl. 435/5

(57) ABSTRACT

The present invention provides methods to assess the genetic safety of stem cells, whether endogenous embryonic stem cells, somatic or adult stem cells, or artificially induced stem cells from non-pluripotent cells, and their differentiated derivatives for use in human medicine, and the applications of modified stem cells to testing environmental or potential genetic or epigenetic modulators such as culture media formulations, substrates or scaffolds, additives, reagents, processes, and processing materials used to prepare stem cells for use.

The present application claims benefit of priority to U.S. Provisional Application Ser. No. 61/178,705, filed May 15, 2009, the entire contents of which are hereby incorporated by reference.

This invention was made with government support under 1R41RR024772-01 awarded by the National Institutes of Health. The government has certain rights in the invention.

### BACKGROUND OF THE INVENTION

1. Field of the Invention

The present invention relates generally to the field of biology. More particularly, it relates to methods to assess the genetic safety of stem cells and their differentiated derivatives for use in human medicine.

2. Description of the Related Art

Embryonic stem (ES) cells, and certain other types of stem cells such as those termed “adult or somatic stem cells” or induced Pluripotent Stem (iPSs) cells, are able to self renew indefinitely while retaining a state of pluripotency that allows them to subsequently differentiate into essentially any of the cell lineages found in the body (West and Daley, 2004; Wagers and Weissman, 2004; Zipori, 2004). Importantly, it is possible to specify the manner in which these cells differentiate while in culture such that a wide variety of different specific types of cells can be derived as needed. In addition, because these cells are maintained in culture, they can be further manipulated by genetic engineering to correct any inherent genetic defect. Most remarkably, once they have been induced to differentiate into a specified cell type in culture, these cells can be used to effect a novel, cell-based approach to therapeutic treatment of otherwise intractable diseases or debilitations, including neurodegenerative diseases such as Parkinson's or Alzheimer's diseases, cardiac disease, diabetes, or various other neuromuscular diseases. Additionally, this approach may be used to treat traumatic injuries including damage to the spinal cord or battlefield injuries in otherwise healthy individuals who represent prime candidates to benefit from the application of regenerative medicine.

In the past, cell-based therapeutic approaches have been used with great success, but only in a very limited range of applications such as bone marrow transplantation (Allan et al., 2007; de Buys et al., 2005), because only very limited types of stem cells (e.g. hematopoetic stem cells) were previously available to be used in this manner. The pluripotency of ES cells has opened the door to applying this approach to treat a much broader range of maladies. This cell-based therapeutic approach holds great promise as an alternative to surgical or drug-based approaches. For this reason, the potential application of this methodology has engendered unprecedented excitement and anticipation in both the biomedical and lay communities.

The function of cells, including their state of differentiation, is based primarily upon the particular subset of genes expressed in each cell type. Essentially, all cells in the body carry the same genetic information. However, different cells express different combinations of genes to develop unique phenotypes. Thus, the proper differentiation and function of a cell is dependent upon its ability to a) maintain its genetic integrity (i.e. keep mutations to a minimum) and b) properly program, via epigenetic mechanisms, the expression of this genetic information to achieve a specified cellular state or phenotype. Few studies have been undertaken to examine the status and/or maintenance of genetic integrity in stem cells or their differentiated derivatives. This is despite the fact that maintenance of proper genetic integrity is critical to cellular differentiation and function. Even if mechanisms of epigenetic programming regulating gene expression remain functionally normal, the occurrence of mutations can lead to aberrant cellular states by either a) interfering with the normal transcription or post-transcriptional processing of genes (regulatory mutations) or b) causing a change in the encoded protein or RNA product of the gene such that it can no longer function normally (structural mutations).

Therefore, the success of the cell-based therapeutic approach is dependent upon maintenance of genetic integrity in these cells, both before and after induction of differentiation to yield specific, functional cell types in vitro, and/or transplantation of differentiated derivatives to mitigate defects in patients in vivo. Aberrancies in the function of stem cells or their derivatives can develop as a result of mutations in key genes regulating cellular proliferation or cell fate. The occurrence of such mutations in stem cells could be particularly problematic because, by their very nature, these cells are destined to give rise to large populations of progeny cells that will undergo differentiation to perform particular functions. Thus, it is important to ensure that the methods involved in deriving, maintaining, manipulating (in vitro), and transplanting (in vivo) stem cells or their derivatives, do not lead to an increase in the frequency or a change in the spectrum of spontaneous mutations. This is especially true regarding the genesis of point mutations, including individual base substitutions or deletions or insertions of small numbers of bases, which represent the most common types of mutations that accrue spontaneously or in response to various mutagenic influences in the environment. Indeed, point mutations are responsible for a majority of inherited genetic diseases in humans (Crow, 2000). Currently, the primary method available to assess the genetic integrity of stem cells or their derivatives is karyotyping. This method detects abnormalities in chromosome number or gross defects in chromosomal structure, but does not detect point mutations. This is despite the fact that point mutations are the most common type of underlying genetic defect leading to specific disease states, including cancer.

Therefore, there is a need for routine methodology for assessing the frequency or spectrum of point mutations in stem cells, their differentiated derivatives, or materials used for their preparation. This need is particularly acute because point mutations are the most common type of underlying genetic defect leading to several specific disease states, including cancer. Herein a novel method to assess the genetic safety of stem cells, their differentiated derivatives for use in human medicine, and applications of transfected stem cells to assess materials safety is provided. This assay will facilitate a highly relevant assessment of genetic integrity that will provide a critically needed level of quality control and safety assurance during the development of protocols and/or materials for use in a variety of different stem cell-based therapeutic approaches and processes. This assay will also facilitate the analysis of the genetic safety of methods of transplantation and subsequent function of transplanted stem cells or their derivatives during pre-clinical testing in animal model systems.

### SUMMARY OF THE INVENTION

In some aspects, the present invention provides methods to assess the genetic safety of stem cells, whether endogenous embryonic stem cells, somatic or adult stem cells, or artificially induced stem cells from non-pluripotent cells, and their differentiated derivatives for use in human medicine, and the applications of transfected stem cells to testing environmental or potential genetic or epigenetic modulators such as culture media formulations, substrates or scaffolds, additives, reagents, processes, and processing materials used to prepare stem cells for use.

In one aspect, the invention provides a method of monitoring mutations in a stem cell or a differentiated derivative of a stem cell comprising: (a) introducing a transgene and a selectable marker into a stem cell line; (b) selecting one or more stem cells comprising one or more transgenes; (c) expanding the one or more transgenic stem cell lines; (d) culturing each transgenic stem cell line to allow one or more spontaneous mutations to accrue; (e) selectively packaging the transgene DNA of the transgenic stem cell line into phage particles and analyzing the resulting phage for mutations; and (f) determining a frequency of mutations and a spectrum of mutations in each mutagenized stem cell line by analyzing the mutant phage.

The stem cell line may be any stem cell line that may be subject to mutation. In some embodiments, the stem cell line is an embryonic stem (ES) cell line, a somatic, tissue-specific or adult stem cell line, or an induced pluripotent stem (iPS) cell line derived from differentiated somatic cells. In other aspects, the stem cell line may derived from any human or non-human stem cell line. In particular embodiments, the non-human cell line may be a primate stem cell line. The stem cell line may be derived from a variety of cell lines. In some embodiments, the stem cell line may be derived from an adult cell line, a somatic cell line, or a non-pluripotent cell line.

The mutation may be any small-scale mutation that may occur in a stem cell. In some embodiments, the mutation is a point mutation. For example, the point mutation may be a base substitution, or a small deletion, addition, or inversion.

The transgene may be any transgene that is capable of functioning as a mutation reporter transgene. The transgene may be introduced into the cell by any method known to one of skill in the art. For the convenience of gene delivery and activation in the cell or organism of interest, the transgene is preferably incorporated into an expression-competent vector, selected from the group consisting of DNA transgene, plasmid, retrotransposon, transposon, jumping gene, viral vector, and a combination thereof. Such vector so obtained may introduced into the cell or organism by a high efficient gene delivery method known to those having skill in the art, including chemical/liposomal transfection, electroporation, transposon-mediated DNA recombination, jumping gene insertion, viral infection, micro-injection, gene-gun penetration, via a plasmid-based delivery vehicle, and a combination thereof. In particular embodiments, the transgene is incorporated into the cell via an expression vector. In particular embodiments, the expression vector is a lambda phage shuttle vector. In particular embodiments, the shuttle vector is an LIZ shuttle vector.

The selective marker may be any known selective marker. In some embodiments, the expression of antibiotic resistance genes may be used to serve as a selective marker for searching of successfully transfected or infected clones, possessing resistance to antibiotics such as penicillin G, ampicillin, neomycin, paromycin, kanamycin, streptomycin, erythromycin, spectromycin, phophomycin, tetracycline, rifapicin, amphotericin B, gentamycin, chloramphenicol, cephalothin, tylosin, or any combination thereof.

In some aspects, the method may further comprise characterizing genomic integration of the transgene in each transgenic stem cell line prior to packaging the genomic DNA into phage particles. In some embodiments, characterizing genomic integration of the transgene comprises: (a) confirming genomic integration of the transgene, (b) determining the number of integrated copies of the transgene in each transgenic stem cell line, (c) analyzing each transgenic stem cell line for an array or dispersed copies of the transgene, and (d) mapping the site(s) of genomic integration of the transgene in each transgenic stem cell line. Confirming the genomic integration of the transgene may be performed by any appropriate method known to those of skill in the art. In some embodiments, confirming genomic integration of the transgene comprises inverse PCR. The number of integrated copies of the transgene may be determined by any appropriate method known to those of skill in the art. In some embodiments, determining the number of integrated copies of the transgene in each stem cell line comprises real-time qPCR. In other embodiments, determining the number of integrated copies of the transgene in each stem cell line comprises Southern blot analysis. Analyzing the transgenic stem cell line for an array or dispersed copy of the transgene may be performed by any appropriate method known to those of skill in the art. In some embodiments, analyzing each transgenic stem cell line for an array or dispersed copies of the transgene comprises a Southern blot analysis. Determining the frequency of mutations and the spectrum of mutations may be performed by any appropriate method known to those of skill in the art. In some embodiments, determining the frequency of mutations and the spectrum of mutations comprises: (a) culturing each transgenic stem cell line to allow spontaneous mutations to accrue, (b) using an assay to determine the frequency of spontaneous mutations and the spectrum of spontaneous mutations in each transgenic stem cell line.

In some apsects, the method further comprises subjecting each transgenic stem cell line to mutagenesis. This may be done, for example, to confirm the efficacy and sensitivity of the assay system. The mutagenesis may be spontaneous or may be artificially induced. Mutagenesis can be caused by any method known to those of skill in the art. In some embodiments, subjecting the stem cell line to mutagenesis comprises exposing the stem cell line to an established mutagen. For example, the established mutagen may be ethylnitrosourea. In other embodiments, the mutagen may be any established mutagen known to induce point mutations.

The stem cell line may be undifferentiated or differentiated to yield specific cell types. The differentiated stem cell line may be differentiated to yield any type of cell and may be differentiated by any method known to those of skill in the art. In some embodiments, the stem cell line is differentiated to form endodermal derivatives, mesodermal derivatives, ectodermal derivatives, or derivatives of the germ line.

In some aspects, the method may further comprise using the transgenic stem cell line to test all aspects and materials, including processes, formulations, reagents or materials, used to culture, cryopreserve or induce differentiation in the stem cells. In further aspects, the method may further comprise using the differentiated transgenic stem cell line to test methods to deliver the stem cells to an intact animal and to monitor differentiated derivatives in the intact animal. The testing may be performed on differentiated or undifferentiated stem cells.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

The term “therapeutically effective” as used herein refers to an amount of cells and/or therapeutic composition (such as a therapeutic polynucleotide and/or therapeutic polypeptide) that is employed in methods of the present invention to achieve a therapeutic effect, such as wherein at least one symptom of a condition being treated is at least ameliorated, and/or to the analysis of the processes or materials used in conjunction with these cells.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 Mutation frequencies and standard errors in somatic cells/tissues and enriched seminiferous tubule cell preparations obtained from 60-day-old male mice. The mutation frequencies are significantly lower for seminiferous tubule cells versus somatic cells (P<0.0001). A test for differences between brain, liver, Sertoli cells (6 days old), and Sertoli cells (8 days old) revealed no significant differences (P=0.1094). Br, brain; Li, liver; SC, Sertoli cells (a somatic cell type in the seminiferous epithelium); SAvg, somatic average; STC, seminiferous tubule cells (a combination of spermatogenic [germ] cells at various stages devoid of somatic Sertoli cells). From Walter et al. (1998).

FIG. 2 Differential accumulation of mutations in pluripotent stem cells and their differentiated derivatives. The frequencies of spontaneous point mutations measured in the lad reporter transgene are graphed as a function of passage number. Frequencies of mutations in pluripotent, undifferentiated ES cells are shown as open circles, and those in hematopoietic cells induced to differentiate from these same ES cells are shown in closed triangles. All are displayed as log values×10⁻⁵ on the Y axis. Note the distinctly rapid increase in the frequency of mutations upon induction of the pluripotent ES cells to differentiate into hematopoietic cells. (Murphey, Hornecker, Cooney, McCarrey, unpublished observations)

FIG. 3 Transgene construct. The Big Blue® Lambda LIZ (LacI/Z) shuttle vector is the bacteriophage lambda vector used in the Big Blue transgenic rodent mutation assay. The vector carries the bacterial lad gene as a mutable target and the a>portion of the lacZ gene as a reporter. The utility of this vector is its ability to act as a target for mutations in vivo that can subsequently be “shuttled” to Escherichia. coli (E. coli) for analysis. The vector is recovered from genomic DNA using Transpack® in vitro lambda packaging extracts available from Stratagene. The pExchange Module EC-Neo is a plasmid cassette, also available from Stratagene. It carries the selectable neo marker gene ligated 3′ to the SV40 promoter (P SV40) that directs ubiquitous expression, followed by a second marker gene (TK) that will not be used in this experiment, and a poly A addition site (pA), all flanked by LoxP sites to facilitate optional excision of the selectable marker gene cassette following integration and selection. The two vectors are fused by blunt-end ligation to either side of a short linker sequence (L).

### DESCRIPTION OF THE ILLUSTRATIVE EMBODIMENTS

A transgene-based assay to determine the frequency and spectrum of point mutations in human and nonhuman primate stem cells and their derivatives is provided. This assay facilitates a highly relevant assessment of genetic integrity that provides a critically needed level of quality control and safety assurance during the development of a variety of different stem cell-based therapeutic approaches and processes that include use of materials.

### I. Purpose of Detecting Mutations

The field of stem-cell-based regenerative medicine is still in its infancy, but is rapidly evolving. Methods and protocols for the derivation and maintenance of pluripotent stem cells in culture, the induction of differentiation of these cells into specific cell types applicable to the treatment human diseases or debilitations, as well as the methods by which these cells will be transplanted into patients and the extent to which they will successfully and safely function following transplantation, are all still under development. Thus, parallel methods to ensure the safety of these methods are also just now beginning to be developed.

The disclosed assay provides a novel system for monitoring the genetic integrity of any type of stem cells during their derivation, maintenance, propagation, and/or differentiation in culture, during cryopreservation, or following their transplantation into recipients/patients during clinical therapeutic applications during preclinical testing in relevant animal model systems. It is critically important that genetic integrity be maintained at optimal levels during the derivation of stem cells and the subsequent induction of differentiation of these cells to produce differentiated cell types as well as during their subsequent use in clinical protocols to treat human diseases or debilitations. In some aspects, the assay provides optimized protocols for 1) delivering a mutation reporter transgene to the cells to be monitored, 2) using a mutation reporter transgene to assess the frequency and spectrum of point mutations in these cells at any stage during the process of their derivation or manipulation, 3) using a mutation reporter transgene to optimize protocols so as to avoid the use of methods or materials that might introduce mutations at a high rate, 4) monitoring genetic safety during preclinical testing in nonhuman primate model systems of methods or materials for use in stem-cell-based therapeutic approaches or processes that will ultimately be applied to human patients. Thus, the assay provides critically important information regarding the genetic safety of cells to be used in therapeutic applications in human patients and of the methods or materials to be used for this purpose. This will facilitate optimization of protocols for clinical use in humans to ensure the maintenance of genetic integrity during these procedures, thus avoiding the inadvertent introduction of mutations that could lead to catastrophic outcomes during subsequent cell-based therapeutic applications. In so doing, this invention will help ensure that stem-cell-based approaches to regenerative medicine will be optimally effective without unexpected or unwanted deleterious outcomes such as the development of tumors from the transplanted cells due to the induction or accumulation of point mutations in these cells.

### II. Methods for Detecting Mutations

In this disclosure, a novel approach to monitoring genetic integrity in stem cells and their derivatives based on the introduction of a lambda phage shuttle vector as a transgene is provided.

In some aspects, the invention provides a method of monitoring mutations in a stem cell line comprising (a) introducing a transgene plus a selectable marker into a stem cell line or freshly isolated stem cells; (b) selecting one or more stem cells carrying one or more transgenes; (c) expanding one or more transgenic stem cell lines; (d) culturing each transgenic stem cell line to allow spontaneous mutations to accrue; (e) selectively packaging the mutation reporter transgene recovered from the transgenic stem cell line into phage particles and analyzing the resulting phage for mutant phenotypes; (f) determining a frequency of mutations and a spectrum of mutations in each stem cell line by analyzing the mutant phage; and (g) using the transgene-containing cells for testing processes or materials used in the handling, culture, storage or delivery of stem cells. In another embodiment, the invention provides a method of monitoring mutations in a stem cell line that was obtained with a transgene.

Transgenic shuttle vectors, especially those based on the prokaryotic lac operon, facilitate a convenient, efficient and accurate assay for mutagenic processes in living cells (Dycaico et al., 1994). The shuttle vector is maintained as an integrated transgene in the cellular genome, such that it is subject to the same mutagenic effects that impact all other genomic loci. This vector can be easily recovered from genomic DNA, packaged into infectious phage particles and used in a colorimetric assay to detect mutational events. Once detected, mutant copies of the transgene can then be recovered and sequenced to determine the specific aberration involved, and, in this way, a population of mutant transgenes can be examined to provide a spectrum of mutations characteristic of any particular cell type or environmental history. Thus, this system provides an ideal approach to monitoring the frequency and spectrum of spontaneous point mutations in stem cells as they are 1) initially derived, 2) maintained indefinitely in an undifferentiated state, 3) stored using various methods, 4) genetically manipulated in any way while in culture (e.g. transfection to add a specific functional gene), 5) induced to initiate differentiation in culture, and/or 6) transplanted into individuals as part of a therapeutic regimen.

Genetic integrity can be characterized in undifferentiated human or nonhuman primate (NHP) ES cells, or in differentiated human or NHP ES cells, or in other somatic or adult stem cells or in iPS cells, following induction to form endodermal derivatives (e.g. pancreatic cells), mesodermal derivatives (e.g. neural cells), or ectodermal derivatives (e.g. skin/fibroblasts) in cell culture. Importantly, the mutation reporter transgene is susceptible to a variety of different mutagens to which the stem cells may be exposed and will therefore facilitate an accurate assessment of mutagenic events in these cells. Thus, the introduction of this transgene into test lots of human stem cells can be used to determine the genetic safety of a particular line of human stem cells, or the use of those cells in a particular derivation, storage or differentiation protocol, or the use of specific materials or reagents in conjunction with specific protocols. In addition, this same approach can be used in clinically relevant nonhuman primate model systems to analyze genetic safety of stem cells or their differentiated derivatives following transplantation into appropriate organs in intact animals. In this regard, the transplanted cells can be sustained, and allowed to propagate and function in vivo in transplanted primates for an extended period of time, then the transplanted cells can be recovered and it can be determined if the methods used to transplant stem cell derivatives into living animals, and/or subsequent exposure of these cells to specific microenvironments in the living animal are, themselves, mutagenic.

This approach is preferable to previously known methods for at least three reasons: 1) it will facilitate an assessment of the frequency and spectrum of point mutations, the most common source of inherited genetic diseases (Crow, 2000), 2) it can be applied relatively easily to any individual stem cell line or to primary or early passage stem cells by using optimized methods to introduce the shuttle vector and subsequently monitor the frequency and spectrum of mutations in the reporter transgene, and 3) the introduction and integration of the shuttle vector will typically impose no effect on the viability or function of the recipient cells, and so it is able to act as a neutral reporter sequence (Kohler et al., 1991). There already exists a very substantial database regarding the frequency and spectrum of point mutations detected by this method in a wide variety of cell types and cell lines (Dycaico et al., 1994; de Boer and Glickman, 1998; Provost et al., 1993; Schaaper and Dunn, 1991). Therefore, this methodology is poised for application as a means to monitor the genetic safety of stem cells at all phases of the process of stem-cell-based regenerative medicine.

This assay provides optimized methodology for introducing the “mutation reporter” transgene into human or nonhuman primate stem cells and using this method to monitor the frequency and spectrum of spontaneous point mutations as stem cells are derived, maintained in an undifferentiated state, stored, or as they are induced to differentiate into specific cell types for therapeutic applications. Further, the assay provides a reliable, standardized assay for the frequency and spectrum of point mutations that can be used to monitor genetic safety at all stages of development, testing, and routine application of stem cell-based therapeutic approaches, processes, or materials in humans or nonhuman primates, or other mammals, including analysis of: 1) undifferentiated stem cells from various sources, 2) various differentiated cell types derived in vitro from stem cells, and 3) stem cell derivatives following transplantation and engraftment in vivo.

### III. Mutations

In some aspects, the present invention provides for a method of monitoring mutations in a stem cell line. These mutations include small-scale mutations, including but not limited to those discussed below.

A. Types of Mutations

Aberrancies in the function of stem cells or their derivatives can develop as a result of mutations in key genes regulating cellular proliferation or cell fate. Small-scale mutations, such as those affecting one or a few nucleotides in a gene, are collectively known as point mutations, including base substitutions, small insertions, or small deletions. These small-scale mutations represent the most common types of mutations that accrue spontaneously or in response to various mutagenic influences in the environment. Indeed, point mutations are responsible for a majority of inherited genetic diseases in humans (Crow, 2000). Large-scale mutations in chromosomal structure include amplifications of repeat sequences, deletions or duplications of large chromosomal regions, chromosomal translocations, deletions, inversions, loss of heterozygosity, aneuploidy or polyploidy.

B. Frequency of Mutations

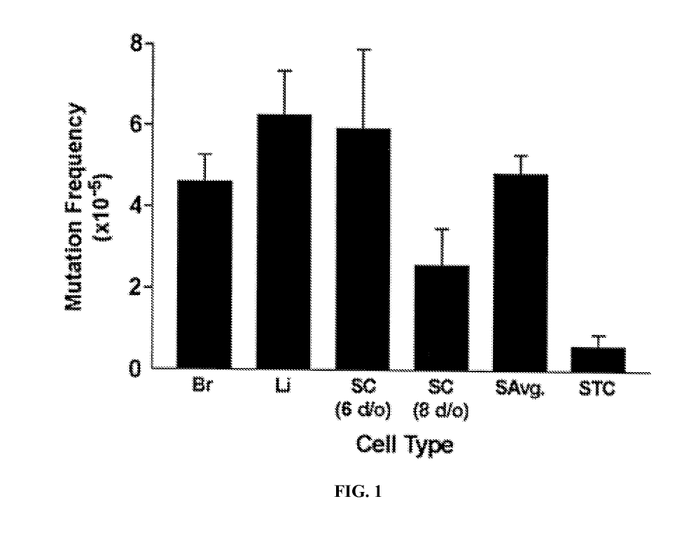

Although all cells of the body contain essentially the same genetic information, the extent to which this genetic information is maintained (genetic integrity) varies in different cell types. For instance, previous studies showed that the frequency of point mutations in male germ cells is significantly lower than that in somatic cells in the same animal (FIG. 1) (Walter et al., 1998). Similarly, other data suggest the same is true in females. This leads to the striking conclusion that maintenance of genetic integrity is regulated by epigenetic mechanisms, and that the stringency of these mechanisms varies in different cell types. This conclusion is further supported by additional findings showing that the status of epigenetic regulation of genetic integrity is subject to reprogramming during the process of cloning by somatic cell nuclear transfer (SCNT) (Murphey et al., 2009). This is important because a frequently proposed scenario for the future application of stem cell-based therapeutics in humans involves generating “patient-specific” embryonic stem cells through the process of therapeutic cloning by SCNT (Yang et al., 2007).

The previous finding that germ cells maintain a significantly lower frequency of spontaneous mutations than somatic cells (FIG. 1) (Walter et al., 1998) suggests that evolution has favored more stringent DNA repair and related mechanisms in germ cells because of the unique role played by these cells in perpetuating the species from generation to generation. Therefore, it was thought that embryonic stem cells should also maintain a relatively low frequency of spontaneous mutations because they also retain the potential to give rise to large populations of progeny cells. Although the frequency of point mutations in ES cells had not been previously determined, other assessments of genetic integrity support the notion that undifferentiated ES cells optimally maintain their genetic integrity. Thus, an analysis of the frequency of loss of heterozygosity at the Aprt locus in murine ES cells indicated a frequency that is 100 to 1000× lower in ES cells than in embryonic somatic cells from the same strain of mice (Cervantes et al., 2002; Stambrook, 2007). Further, studies of mice carrying a knockout mutation at the p. 53 locus suggest that a p53-mediated pathway specifically contributes to the heightened maintenance of genetic integrity in murine ES cells (Corbet et al., 1999; Xu, 2005). While these studies supported the notion that mechanisms normally function in ES cells to stringently maintain genetic integrity, the methods used to achieve these results were laborious and impractical as a routine approach to assess genetic integrity in a wide variety of ES or other stem cell lines or their derivatives.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Introduce a Lambda Shuttle Vector Transgene into Existing Lines of a) Human, and b) Rhesus Monkey or Baboon Embryonic Stem Cells

Introducing the Big Blue® lambda LIZ (lacI/Z) shuttle vector (commercially available from Stratagene, La Jolla, Calif.) into human, rhesus monkey or baboon ES cell lines allows subsequent monitoring of the frequency and spectrum of point mutations in each line. This is done in the following manner:
(a) Construct transgene—Modify the commercially available lambda shuttle vector to add a floxed selectable marker (Neo).
(b) Introduce the transgene into stem cell lines by electroporation.
(c) Select for cells carrying integrated transgenes.
(d) Clonally expand lines from individual positive colonies from each line.

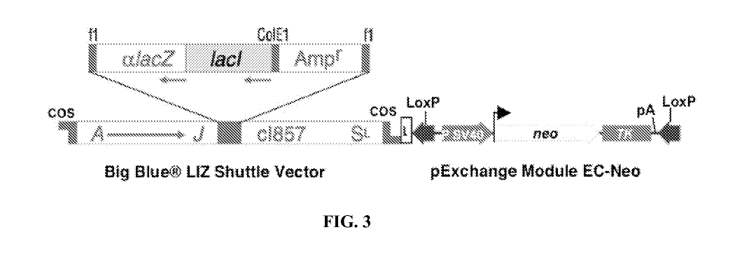

Methods—The strategy and methods used for transforming ES cells with the lambda shuttle vector are derived using detailed protocols described in Nagy et al. (2003). The Big Blue shuttle vector is purchased from Stratagene and modified to facilitate positive selection of cells in which the transgene has become integrated into the genome. This is done by ligating a selectable marker gene to the shuttle vector. A commercially available cassette that includes the neo gene directed by the SV40 promoter is used. Recipient cells that have incorporated the neo gene into their genomes are resistant to the selective drug G418. A schematic representation of the final transgene construct is shown in FIG. 3.

Lines of human ES cells (hES cells) approved for use in research projects supported by federal funding are available from a variety of sources. Based on information at the WiCell website (available on the world wide web at wicell.org/index.php?option=com_oscommerce&Itemid=130), 9 of the 21 approved lines of hES cells are currently available. The three most utilized of the available hES lines are H1 (WiCell), H9 (WiCell) and hES3 (ESI). Each of these lines has been studied extensively (references listed on WiCell website) and certified on the bases of gene expression profiling, karyotype analysis, immunocytochemical analysis and absence of contamination by mycoplasma (WiCell website). In addition, protocols are provided on the WiCell website for handling, maintenance and manipulation of each of these lines.

Linearized transgene DNA is purified using either DNA purification columns (Qiagen) or drop dialysis. Exponentially growing ES cells are re-suspended in electroporation buffer at a density of ˜1×10⁷ cells/ml. Twenty five (25) μg of linearized transgene construct is added to an electroporation cuvette containing 0.8 ml of cell suspension, allowed to stand for 5 minutes, then pulsed with 230 to 250 Volts, 500 μF. After pulsing, the cells are transferred to fresh culture dishes containing mitotically inactivated MEF (mouse embryonic fibroblast) feeder layers, and cultured for 24 hours in non-selective medium. Electroporated cells are then refed with medium containing 200 μg/ml G418 selection drug and incubated for 8 to 10 days, with medium changed every day. Only those cells that have taken up the transgene, and, hence, the selectable neo marker, will survive in these conditions.

Following selection, individual colonies of surviving ES cells are transferred to 96-well dishes containing neo-resistant feeder cells and cultured for 3 to 4 days to expand each colony. At this stage, a portion of each colony is recovered for initial DNA analysis to confirm transgene integration (see Example 2). The remainder of each colony is stored until transgene integration has been confirmed. Those colonies containing integrated transgene are then expanded further to provide sufficient material for the additional analysis described in Examples 2, 3 and 4.

### Example 2

### Confirm and Characterize Genomic Integration of the Transgene in Each Derived ES or Other Stem Cell Line

The genomic integration of the transgene into the ES or other stem cell line may be confirmed and characterized. This step may comprise: a) confirming genomic integration of the transgene, b) determining the number of integrated copies of the transgene in each line, c) determining if multiple copies are present in tandem arrays or dispersed, and d) mapping the site(s) of genomic integration of the transgene in each line. Only those cells with transgenes integrated into the genome will be useful for subsequent experiments.

Methods—Initial screening for genomic integration is based on an inverse polymerase chain reaction (PCR) approach. In one experiment, genomic DNA from a portion of each ES cell colony that survives G418 selection is digested with a restriction enzyme that cuts in a region 0.5 to 1.0 kb from the left end of the transgene construct shown in FIG. 3. A second experiment is conducted with a different restriction enzyme that cuts 0.5 to 1.0 kb inside the right end of the transgene construct. In each case, the fragments yielded by this digestion include a portion of the transgene plus a portion of flanking genomic sequence (the extent of which is determined by the position of the next digestion site for the particular restriction endonuclease(s) used). This is followed in each case by a ligation procedure that yields, among other products, circular molecules carrying the known portion of the transgene plus the unknown genomic flanking sequence. A PCR reaction is then performed using primers complementary to adjacent sites within the known transgene sequence, but facing away from one another. This amplifies the known portion of the transgene sequence plus the unknown portion of flanking genomic sequence. DNA sequencing of the amplification product yields the specific sequence of the flanking genomic sequence, and that sequence is used to interrogate either the human genome database or the rhesus monkey genome database to locate the relevant genomic region. The presence of genomic sequence adjacent to each end of the transgene confirms genomic integration. The genomic sequence flanking the left end of the transgene should normally be immediately adjacent to that flanking the right end of the transgene in the wild-type genome, thus revealing the exact site of genomic integration of the transgene in each case. Potentially confounding results from integration of tandem arrays of transgene are avoided by selecting restriction enzymes that yield very large, unamplifiable products from digestion of adjacent copies of the transgene. Recovery of more than one genomic flanking sequence at each end of the transgene indicates multiple integration sites (see below).

Real-time qPCR is used to estimate the number of copies of transgene per genome in each line of cells in which genomic integration of transgene has been confirmed. A standard curve is generated as a quantitation control by mixing genomic DNA from known numbers of cells with known amounts of transgene.

Southern blot analysis is conducted to further confirm genomic integration and single or multiple integration events in each cell line. Electrophoresed genomic DNA from each transgenic ES cell line is transferred to hybridization membrane and probed with a labeled copy of a portion of the transgene. Evidence of hybridizing fragments corresponding to more than one contiguous region of the genome indicates multiple integration sites.

### Example 3

### Determine the Baseline Frequency and Spectrum of Point Mutations in Each Transgenic ES or Other Stem Cell Line Following >20 Passages in Culture

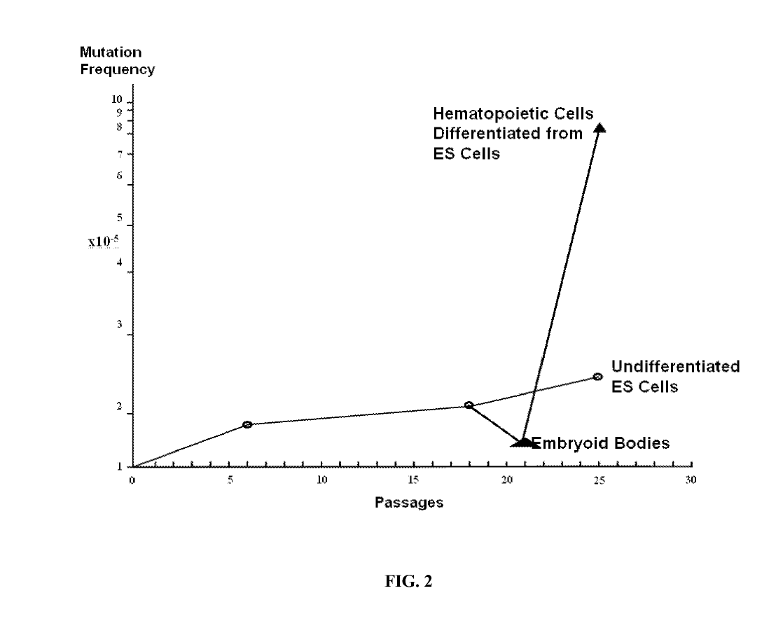

Each line may be cultured for sufficient time to allow spontaneous mutations to accrue, then the “Big Blue” assay is used to determine the baseline frequency and spectrum of acquired spontaneous point mutations in each line of undifferentiated transgenic embryonic stem cells, similar to the analysis of undifferentiated mouse ES cells shown in FIG. 2.

Methods—Standard methods for assessing mutation frequency and spectrum in the transgene are used. Briefly, at least 10⁶ cells from each line of transgenic ES cells that has been passaged 20 or more times in an undifferentiated state are used to generate high molecular weight genomic DNA using the RecoverEase® kit from Stratagene, and that DNA is then subjected to a lambda phage packaging reaction using Transpack packaging extract (Stratagene) to selectively incorporate the transgenes (but not any of the cellular genomic DNA) into infectious phage particles. The phage is then used to infect host E. coli cells in the presence of IPTG and X-gal such that any resulting phage plaque carrying a mutated lacI repressor gene recovered from the mutation reporter transgene is blue due to production of beta-galactosidase, whereas plaques containing non-mutated copies of the lacI gene fail to express beta-galactosidase and are clear. Blue plaques are picked, confirmed by replating, and then subjected to DNA sequencing to further confirm the mutation and to determine the specific change in DNA sequence. The frequency of point mutations in each line is determined by dividing the number of confirmed, individual mutation events recovered from each stem cell line by the total number of plaques examined. Comparisons of frequencies among each of the transgenic stem cell lines are analyzed for statistical significance. DNA sequence information for the lacI gene from each mutant plaque is used to derive the spectrum of point mutations in each stem cell line examined. For each mutation, the position within the transgene, the exact change in base sequence, and the category of change relative to the wild-type sequence of the lacI gene including small deletions, small insertions, or base substitutions including transitions or transversions are determined.

### Example 4

### Confirm that the Integrated Transgene is Susceptible to Mutagenesis in Each Transgenic Stem Cell Line

To confirm the efficacy of the mutation reporter transgene system, each transgenic stem cell line may be subjected to mutagenesis using the established mutagen, ENU (ethylnitrosourea), then the frequency and spectrum of mutations can be determined in the mutagenized cells. Studies show that ENU typically induces point mutations (Favor, 1999). This validates that the integrated transgene is susceptible to an exemplary type of mutagenesis and represents a model that supports the utility of this approach for testing specific cell lines or processes and materials involved in stem cell-base therapeutic applications.

Methods—Protocols are well established for using ENU to induce point mutations in cultured cells (van Zeeland et al., 1985). Stock solutions of ENU are prepared at 25 to 50 mM in phosphate buffered saline (pH=6). Approximately 10⁸ cells/sample are suspended in 2.5 ml culture media and treated for 1 hour with 1 to 10 mM ENU. The cells are then washed and cultured for 1 to 2 passages. High molecular weight genomic DNA is then recovered from the surviving cells and analyzed for the frequency and spectrum of point mutations in the transgene as described in Example 3. The significance of any differences in mutation frequency or spectrum observed between ENU-treated and non-treated samples from each transgenic stem cell line is assessed by statistical analysis as performed with the mutation frequency and spectrum data as previously described (Caperton et al., 2007).

### Example 5

### Results

### Use of the Lambda Shuttle Vector to Assess Genetic Integrity in Germ Cells and Somatic Cells of Transgenic Mice

The “Big Blue Mouse” transgenic system was previously used to assess the frequency of mutations in germ cells and somatic cells of prepubertal, pubertal and adult male mice (Walter et al., 1998). As shown in FIG. 1, it was found that the frequency of spontaneously acquired point mutations in germ cells was significantly lower than that in somatic cells at all ages tested. Furthermore, an unexpected decrease in mutation frequency was observed as spermatogenic cells differentiated from pre-meiotic spermatogonia to meiotic spermatocytes. Interestingly, this decrease correlates with a period when spermatogenic cells are known to undergo significant apoptosis, suggesting that spermatogonia carrying relatively higher frequencies of mutations are non-randomly included among the cells that undergo apoptosis, leaving those spermatogonia carrying relatively lower frequencies of mutations to continue to differentiate through the process of spermatogenesis. These results document tissue-specific differences in mutation frequencies, thus demonstrating epigenetic regulation of genetic integrity.

Use of the lambda shuttle vector to assess the kinetics of acquisition of spontaneous mutations during the development of the germ line and soma, respectively. A study to chronicle the frequency of mutations during development of somatic and germ cell lineages in males and females was conducted. Results compiled to date from previously published studies as well as from ongoing studies show that female germ cells isolated from neonatal pups at 2-6 days postpartum maintain relatively low frequencies of spontaneous mutations similar to that seen in male germ cells (Murphey, MacMahan, Walter, McCarrey, unpublished data). This study confirms the differential regulation of genetic integrity between germ cells and somatic cells throughout development and into postnatal life. This, in turn, further documents the activity of cell-type specific epigenetic mechanisms regulating genetic integrity.

Use of the Lambda Shuttle Vector to Assess Genetic Integrity in Fetuses Produced by Assisted Reproductive Technologies.

The same transgenic mouse system was used to compare mutation frequencies in mid-gestation fetuses produced by methods of assisted reproductive technologies (ARTs) with those produced by natural reproduction (Caperton et al., 2007). As shown in Table 1, it was found that the frequency and spectrum of point mutations in fetuses produced by ARTs did not differ significantly from that in naturally conceived fetuses.

TABLE 1
Mutant Frequencies in Midgestation Fetuses

| Method of | Culture | Stage at | No. | Total | Mutant | Conf. Ind. | Mut. Freq. ± |
|---|---|---|---|---|---|---|---|
| Conception | Media | Transfer | Fetuses | pfu^{a} | Plaques^{b} | Mutants^{c} | SE (× 10⁻⁵)^{d} |
| Natural | None | n/a | 15^{e} + 5^{f} | 4,642,208 | 59 | 50 | 1.08 ± 0.15 |
| Natural | CZB | 2-cell | 15^{e} | 2,339,815 | 31 | 31 | 1.32 ± 0.24 |
| Natural | CZB | Mor/Blast | 15^{e} | 2,418,751 | 23 | 21 | 0.87 ± 0.19 |
| Natural | WM | Mor/Blast | 15^{e} | 2,774,900 | 33 | 33 | 1.19 ± 0.21 |
| IVF | CZB | Mor/Blast | 5^{f} | 2,210,684 | 22 | 21 | 0.95 ± 0.21 |
| ICSI | CZB | Mor/Blast | 5^{f} + 16^{g} | 8,081,035 | 106 | 85 | 1.05 ± 0.11 |
| ROSI | CZB | Mor/Blast | 5^{f} | 2,312,684 | 24 | 23 | 0.99 ± 0.21 |

^{a}pfu = “plaque-forming units”,
^{b}Total mutant plaques recovered.
^{c}Confirmed independent mutants (= total mutants recovered minus potential “jackpot mutants”).
^{d}Mutation frequency based on confirmed independent mutants.
^{e}Three fetuses each from five different parental pairs.
^{f}One fetus each from five different parental pairs.
^{g}16 additional fetuses from three different parental pairs. (See Caperton et al., 2007, Appendix 2 for further details).

Use of the Lambda Shuttle Vector to Assess Genetic Integrity in Fetuses Produced by Somatic Cell Nuclear Transfer (SCNT) (Cloning).

Frequencies of mutations in mouse fetuses produced by cloning are examined (Murphey et al., 2009). As shown in Table 2, the frequency of mutations in populations of three different donor cell types, adult fibroblasts, adult cumulus cells, and fetal neuronal cells was determined. Mutation frequencies were significantly different in each of these donor cell populations. However, when mid-gestation fetuses produced by SCNT using each of the different donor cell types were examined, consistently low mutation frequencies were found that did not differ in clones produced from each of the different donor cell types. This result leads to two major conclusions: 1) that there is a “bottleneck effect” during cloning that tends to restrict transmission of acquired spontaneous mutations due to the fact that each cloned fetus is derived from a single donor cell and it is statistically unlikely any one cell will carry a spontaneous mutation in the reporter transgene given the low frequency of such mutations in each donor cell population, and 2) that epigenetic mechanisms that regulate genetic integrity undergo reprogramming during the cloning (SCNT) process as evidenced by the fact that mid-gestation fetuses produced by cloning from somatic cells in which frequencies of mutations are significantly higher than those in germ cells are not significantly different than those in mid-gestation fetuses produced by natural fertilization.

TABLE 2
Mutation Frequencies in Donor Cell Types and Cloned Fetuses

| | | | Mutant | Mut. Freq. ± |
|---|---|---|---|---|
| | Tissue type | Total pfu | plaques | SE (×10⁻⁵) |
| Donor | Fetal | 2,776,250 | 52 | 1.01 ± 0.26 |
| Cells | neuronal | | | |
| | Cumulus | 860,750 | 35 | 2.86 ± 0.69 |
| | Fibroblast | 423,000 | 30 | 3.87 ± 1.29 |
| Cloned | Fetal | 1,239,469 | 11 | 0.99 ± 0.27 |
| Embryos | neuronal | | | |
| | Cumulus | 1,117,000 | 10 | 0.99 ± 0.28 |
| | Fibroblast | 1,128,340 | 11 | 0.97 ± 0.29 |

pfu = “plaque-forming units”,
Mutant plaques = total mutants recovered minus potential “jackpot mutants”.
Mutation frequency based on confirmed independent mutants

TABLE 3
Mutation Frequencies in Mouse ES Cells vs Somatic Cells

| | | Mut. Freq.^{a} | Somatic | Stage/ | Mut. Freq.^{a} |
|---|---|---|---|---|---|
| ES Cell Line | Passage | (×10⁻⁵) | Tissue | Age | (×10⁻⁵) |
| 30A | 6 | 1.8 | Embryo | 10.5 dpc^{b} | 1.3 |
| | 18 | 2.18 | Liver | 15.5 dpc^{b} | 2.82 |
| | 25 | 2.36 | Liver | 6.0 dpp^{c} | 6.20 |

Use of the Lambda Shuttle Vector to Analyze the Differential Accumulation of Mutations in Pluripotent Stem Cells and their Differentiated Derivatives.

Data showing that undifferentiated ES cells do maintain a relatively low frequency of spontaneous mutations over many passages in culture (FIG. 2) was obtained. Upon induction of differentiation of these cells (i.e., to form hematopoietic cells), the frequency of mutations rapidly increased by approximately four-fold (FIG. 2). This result illustrates that pluripotent cells maintain genetic integrity more stringently than differentiated cells. It also demonstrates the efficacy of the mutation reporter transgene approach for analyzing the genetic safety of different cells types.

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Allan et al., Curr. Opin. Cardiol., 22:104-110, 2007.
Caperton et al., Proc. Natl. Acad. Sci. USA, 104:5085-5090, 2007.
Cervantes et al., Proc. Natl. Acad. Sci. USA, 99:3586-3590, 2002.
Corbet et al., Oncogene, 18:1537-1544.
Crow, Nat. Rev. Genet., 1:40-47, 2000.
de Boer and Glickman, Genetics, 148:1441-51, 1998.
de Buys et al., Autoimmun Rev., 4:442-449, 2005.
Dycaico et al., Mut. Res., 307:461-478, 1994.
Favor, Mut. Res., 428:227-236, 1999.
Kohler et al., Proc. Natl. Acad. Sci. USA, 88:7958-7962, 1991.
Murphey Mut. Res., 106:4731-4735, 2009.
Nagy et al., In: Manipulating the Mouse Embryo: A Laboratory Manual., 3^{rd} Ed., Cold Spring Harbor Lab. Press, Cold Spring Harbor, N.Y., 2003.
Provost et al., Mut. Res., 288:133-49, 1993.
Schaaper and Dunn, Genetics, 129:317-326, 1991.
Stambrook, Mech. Ageing Devel., 128:31-35, 2007.
van Zeeland et al., Env. Health Pers., 62:163-169, 1985.
Wagers and Weissman, Cell, 116:639-648, 2004.
Walter et al., Proc. Natl. Acad. Sci. USA, 95:10015-10019, 1998.
West and Daley, Bone Marrow Transpl., 33:135, 2004.
Xu, Cell Cycle, 4:363-364, 2005.
Yang et al., Nat. Genet., 39:295-302, 2007.
Zipori, Nat. Rev. Genet., 5:873-878, 2004.

1-29. (canceled)

30. A method of monitoring mutations in a stem cell or a differentiated derivative of a stem cell comprising:
(a) introducing a transgene and a selectable marker into a stem cell line;
(b) selecting one or more stem cells comprising one or more transgenes;
(c) expanding the one or more transgenic stem cell lines;
(d) culturing each transgenic stem cell line to allow one or more spontaneous mutations to accrue;
(e) selectively packaging the transgene DNA of the transgenic stem cell line into phage particles and analyzing the resulting phage for mutations; and
(f) determining a frequency of mutations and a spectrum of mutations in each mutagenized stem cell line by analyzing the mutant phage.

31. The method of claim 30, wherein the stem cell line is an embryonic stem (ES) cell line, a somatic, tissue-specific or adult stem cell line, or an induced pluripotent stem (iPS) cell line derived from differentiated somatic cells.

32. The method of claim 30, wherein the mutation is a point mutation.

33. The method of claim 32, wherein the point mutation is a base substitution, or a small deletion, addition, or inversion.

34. The method of claim 30, wherein the expression vector is a lambda shuttle vector comprising a mutation reporter transgene.

35. The method of claim 30, wherein the stem cell line is a human cell line.

36. The method of claim 30, wherein the stem cell line is derived from an adult cell line, a somatic cell line, or a non-pluripotent cell line.

37. The method of claim 30, wherein the stem cell line is a non-human cell line.

38. The method of claim 37, wherein the non-human cell line is a primate stem cell line.

39. The method of claim 30, wherein introducing the expression vector comprises a gene delivery method selected from the group consisting of a plasmid-based delivery vehicle, electroporation, liposomal transfection, chemical transfection, transgenic DNA recombination, viral infection, transposon insertion, jumping gene insertion, micro-injection, gene-gun penetration, and a combination thereof.

40. The method of claim 30, further comprising characterizing genomic integration of the transgene in each transgenic stem cell line prior to packaging the genomic DNA into phage particles.

41. The method of claim 40, wherein characterizing genomic integration of the transgene comprises:
a) confirming genomic integration of the transgene,
b) determining the number of integrated copies of the transgene in each transgenic stem cell line,
c) analyzing each transgenic stem cell line for an array or dispersed copies of the transgene, and
d) mapping the site(s) of genomic integration of the transgene in each transgenic stem cell line.

42. The method of claim 41, wherein confirming genomic integration of the transgene comprises inverse PCR.

43. The method of claim 41, wherein determining the number of integrated copies of the transgene in each stem cell line comprises real-time qPCR or Southern blot analysis.

44. The method of claim 41, wherein analyzing each transgenic stem cell line for an array or dispersed copies of the transgene comprises a Southern blot analysis.

45. The method of claim 30, wherein determining the frequency of mutations and the spectrum of mutations comprises:
a) culturing each transgenic stem cell line to allow spontaneous mutations to accrue;
b) using an assay to determine the frequency of spontaneous mutations and the spectrum of spontaneous mutations in each transgenic stem cell line.

46. The method of claim 30, further comprising subjecting each transgenic stem cell line to mutagenesis.

47. The method of claim 46, wherein subjecting the stem cell line to mutagenesis comprises exposing the stem cell line to an established mutagen.

48. The method of claim 47, wherein the established mutagen is ethylnitrosourea.

49. The method of claim 30, wherein the stem cell line is undifferentiated or differentiated to yield specific cell types.

50. The method of claim 49, wherein the stem cell line is undifferentiated.

51. The method of claim 49, wherein the stem cell line is differentiated to yield specific cell types.

52. The method of claim 51, wherein the stem cell line is differentiated by induction to form endodermal derivatives, mesodermal derivatives, ectodermal derivatives, or derivatives of the germ line.

53. The method of claim 30, further comprising using the transgenic stem cell line to test processes, formulations, reagents or materials used to culture, cryopreserve or induce differentiation in the stem cells.

54. The method of claim 30, further comprising using the differentiated transgenic stem cell line to test methods to deliver the stem cells to an intact animal and to monitor differentiated derivatives in the intact animal.

55. The method of claim 30, further comprising differentiating the transgenic stem cell line.

56. The method of claim 30, further comprising using the differentiated transgenic stem cell line to test processes, formulations, reagents or materials used to culture, cryopreserve or induce differentiation in the stem cells.

57. The method of claim 55, further comprising using the differentiated transgenic stem cell line to test methods to deliver the stem cells to an intact animal and to monitor differentiated derivatives in the intact animal.

58. A method of monitoring mutations in a stem cell or a differentiated derivative of a stem cell comprising:
(a) obtaining a stem cell line containing a transgene and a selectable marker;
(b) selecting one or more stem cells comprising one or more transgenes;
(c) expanding the one or more transgenic stem cell lines;
(d) culturing each transgenic stem cell line to allow one or more spontaneous mutations to accrue;
(e) selectively packaging the transgene DNA of the transgenic stem cell line into phage particles and analyzing the resulting phage for mutations; and
(f) determining a frequency of mutations and a spectrum of mutations in each mutagenized stem cell line by analyzing the mutant phage.
